(19) United States
(12) Patent Application Publication
(10) Pub. No.: US 2024/0285576 A1
(43) Pub. Date: Aug. 29, 2024

(54) R-MDMA FOR TREATMENT OF PAIN

(71) Applicant: Mind Medicine, Inc., New York, NY (US)

(72) Inventor: Gennady N. SMAGIN, Newark, DE (US)

(73) Assignee: Mind Medicine, Inc., New York, NY (US)

(21) Appl. No.: 18/421,264

(22) Filed: Jan. 24, 2024

Related U.S. Application Data
(60) Provisional application No. 63/487,370, filed on Feb. 28, 2023.

(51) Int. Cl. A61K 31/36; A61K 45/06; A61P 25/04; A61P 29/00

(52) U.S. Cl. CPC A61K 31/36; A61K 45/06; A61P 25/04; A61P 29/00

(57) ABSTRACT

A method of treating pain, by administering an effective amount of a composition of MDMA, enantiomers thereof (R-MDMA, S-MDMA), a unique mixture of enantiomers (not 1:1), MDMA-like compounds, prodrugs of MDMA, enantiomers, or MDMA-like compounds, analogs thereof, derivatives thereof, or salts thereof to an individual, and treating pain in the individual.

### BACKGROUND OF THE INVENTION

### 1. Technical Field

The present invention relates to compositions and methods for treating pain.

### 2. Background Art

People feel pain when nerves detect tissue or nerve damage and/or real or perceived bodily harm and transmit information about the damage to the brain. Acute pain is usually temporary and felt with an injury or surgery and treating the injury can relieve the pain. Chronic pain can last much longer than acute pain (weeks, months, or years), and can be continuous or intermittent and stopping for periods of time. The clinical definition of chronic pain is pain that lasts longer than 6 months and can continue when the injury or illness has been treated. Examples of chronic pain include arthritis, migraine headaches, cancer pain, neuropathic pain, diabetic neuropathy, chemotherapy-induced neuropathy, phantom limb pain, back pain, pain from fibromyalgia and nerve pain. Chronic pain can also cause emotional effects in an individual, such as depression, anger, anxiety, and fear of re-injury. Pain can be classified according to the affected anatomy (visceral, neurological, osteo-articular) or the mechanism responsible for it (nociceptive due to tissue injury, neuropathic due to damage to the nerves or nervous system, inflammatory due to abnormal inflammation, and functional pain without obvious origin).

Pain is currently treated with either non-steroidal anti-inflammatory drugs (NSAIDS), opioids or antidepressants such as selective serotonin reuptake inhibitors (SSRIs). NSAIDS such as aspirin, ibuprofen, ketoprofen, naproxen, and celecoxib work by blocking Cox-1 and Cox-2 enzymes which help make prostaglandins in the body. Prostaglandins are released by damaged tissue and increase the feeling of pain, so by reducing the amount of prostaglandins, less pain is felt. Using NSAIDs regularly to treat pain can lead to ulcers in the esophagus, stomach, and small intestine, cause damage to the kidneys, as well as increased risk of heart attacks and stroke. Opioids produce pain relief as well as euphoria by binding and activating opioid receptors on nerve cells involved in feeling pain. When attached to receptors, opioids block signals from the brain and release dopamine in the body. Opioids include morphine, fentanyl, oxycodone hydrochloride, hydrocodone and acetaminophen, oxycodone and acetominophen, among others. Opioid overuse can lead to addiction.

3,4-Methylenedioxymethamphetamine (MDMA) is a psychoactive drug that alters mood and perception, and is investigated as an adjunct in psychotherapy for posttraumatic stress disorder (PTSD), social anxiety, autism spectrum disorder (Danforth, 2016; Danforth et al., 2018; Danforth et al., 2016; Mithoefer et al., 2019; Mithoefer et al., 2010; Oehen et al., 2013), and may later also be studied and used for a range of other medical conditions. Such conditions where MDMA or related substances may be useful include, but is not limited to, substance-use disorder, depression, anxiety disorder, anxiety with life-threatening disease, personality disorder including narcistic and antisocial disorder, and obsessive-compulsive disorder. MDMA or related substances can also be used to enhance couple therapy. MDMA has not been used in treating pain.

MDMA is a racemic substance containing equal amounts of the enantiomers S(+)- and R(−)-MDMA. Preclinical research indicates that S-MDMA mainly releases dopamine (DA), norepinephrine (NE), serotonin (5-HT), and oxytocin while R-MDMA may act more directly on serotonin 5-HT2A receptors and release prolactin (PRL). Animal studies also indicate that the two enantiomers act synergistically to produce the subjective effects of MDMA and that S-MDMA is mainly responsible for psychostimulation while R-MDMA may have fewer adverse effects and have greater prosocial effects. However, acute effects of separate enantiomers, S- and R-MDMA have never been validly examined in a human study.

MDMA and related substances are thought to produce positive therapeutic long-term effects in the context of MDMA/substance-assisted psychotherapy by producing acute subjective positive mood effects that also enhance the effectiveness of psychotherapy and can be beneficial on their own. Such acute beneficial MDMA-effects include, but are not limited to, feelings of well-being, feelings of connectivity to others, feelings of increased trust, feelings of love, enhanced emotional empathy, and enhanced feelings of pro-sociality and prosocial behavior (Hysek et al., 2014; Liechti et al., 2001; Schmid et al., 2014; Vollenweider et al., 1998a).

There remains a need for an effective treatment for pain.

### SUMMARY OF THE INVENTION

The present invention provides for a method of treating pain, by administering an effective amount of a composition of MDMA, enantiomers thereof (R-MDMA, S-MDMA), a unique mixture of enantiomers (not 1:1), MDMA-like compounds, prodrugs of MDMA, enantiomers, or MDMA-like compounds, analogs thereof, derivatives thereof, or salts thereof to an individual, and treating pain in the individual.

### DESCRIPTION OF THE DRAWINGS

Other advantages of the present invention are readily appreciated as the same becomes better understood by reference to the following detailed description when considered in connection with the accompanying drawings wherein:

FIG. 1 is an illustration of a mouse tail flick test;

FIG. 2 is a graph of mouse tail flick response (latency) over time;

FIG. 3 is a graph of percent MPE, mouse tail flick response over time;

FIG. 4 is an illustration of the Hargreaves method; and

FIG. 5 is a graph of the effect of R-MDMA in rat thermal hyperalgesia test as measured by the Hargreaves test.

### DETAILED DESCRIPTION OF THE INVENTION

The present invention provides for a method of treating pain, by administering an effective amount of a composition of MDMA, enantiomers thereof (R-MDMA, S-MDMA), a unique mixture of enantiomers (not 1:1), MDMA-like compounds, prodrugs of MDMA, prodrugs of enantiomers, or MDMA-like compounds, analogs thereof, derivatives thereof, hydrates thereof, or salts thereof to an individual, and treating pain.

“Pain” as used herein can refer to any discomfort in the body. The pain can be the general types of acute (such as injury or paper cut), chronic, nociceptive (such as post-surgical pain, visceral, somatic, or radicular), neuropathic, inflammatory, or functional. Chronic pain can be further classified as chronic primary pain (characterized by disability or emotional distress and not better accounted for by another diagnosis of chronic pain) or chronic secondary pain (such as chronic cancer-related pain, chronic post-surgical or post-traumatic pain, chronic neuropathic pain, diabetic neuropathy, chemotherapy-induced neuropathy, chronic secondary headache or orofacial pain, chronic secondary visceral pain, or chronic secondary musculoskeletal pain).

The pain can be caused from a physical state in the body (such as injury, damaged tissue, surgery, cancer or cancer breakthrough, diabetes, migraines or other headaches, arthritis, fibromyalgia, back pain, nerve pain, shingles, radiation, or chemotherapy drugs) as well as an emotional state (such as anxiety or depression).

MDMA is an amphetamine derivative which, unlike prototypical amphetamines, predominantly enhances serotonergic neurotransmission via release of 5-HT through the SERT and it less potently also releases dopamine and norepinephrine through the dopamine transporter (DAT) and norepinephrine transporter (NET), respectively (Hysek et al., 2014b; Verrico et al., 2007). Furthermore, MDMA is known to trigger oxytocin release which may contribute to its effects to increase trust, prosociality, and enhanced empathy. MDMA is therefore referred to as an “entactogen” or “empathogen”.

MDMA-like compounds can include 3,4-methylenedioxyamphetamine (MDA), 3,4-methylenedioxyethylamphetamine (MDEA), 1-(1,3-benzodioxol-5-yl)methyl-2-butanamine (MBDB), 1-(1,3-benzodioxol-5-yl)-2-aminobutane (BDB, also known as MDB) methylone, ethylone, 5,6-methylenedioxy-2-aminoindane (MDAI), 5-iodo-2-aminoindane (5-IAI), 4-(2-aminopropyl)-benzofuran (4-APB), 5-(2-aminopropyl)-benzofuran (5-APB), 6-(2-aminopropyl)-benzofuran (6-APB), N-methyl-1-(2,3-dihydrobenzofuran-5-yl)-propan-2-amine (5-MAPDB), 6-(2-methylaminopropyl)-benzofuran (6-MAPB), or other compounds, namely a benzofuran, aminoindane or cathinone or mixed dopaminergic-serotonergic amphetamine and their N-alkylated analogs, with an MDMA-like pharmacological profile (Rickli et al., 2015a; Rickli et al., 2015b; Simmler et al., 2013) or active metabolites of such substances (Luethi et al., 2019). There is similarity of the structures of the MDMA-like compounds: all the compounds contain a 3,4-substitution of the benzene ring in the phenethylamine structure which is typical for MDMA-like compounds that preferably act on serotonin versus dopamine transporters to primarily release serotonin. Any active metabolites can also be used.

Compounds can be used in any suitable pharmaceutical salt form such as hydrochloride or dimesylate, Salts can include acid-addition salts and base-addition salts. The acid that is added to the compound to form an acid-addition salt can be an organic acid or an inorganic acid. A base that is added to the compound to form a base-addition salt can be an organic base or an inorganic base.

The salt can be a metal salt made from the addition of an inorganic base to the compounds herein. The inorganic base consists of a metal cation paired with a basic counterion, such as, for example, hydroxide, carbonate, bicarbonate, or phosphate. The metal can be an alkali metal, alkaline earth metal, transition metal, or main group metal. The metal can be lithium, sodium, potassium, cesium, cerium, magnesium, manganese, iron, calcium, strontium, cobalt, titanium, aluminum, copper, cadmium, or zinc. Therefore, the metal salt can be a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can be made from the addition of ammonia or an organic amine to the compounds. The organic amine can be trimethyl amine, triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrazole, pyrazolidine, pyrazoline, pyridazine, pyrimidine, imidazole, or pyrazine. Therefore, salt can be a triethyl amine salt, trimethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrazole salt, a pyridazine salt, a pyrimidine salt, an imidazole salt, or a pyrazine salt.

Acid addition salts can be made from the addition of an acid to the compound. The acid can be hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisic acid, gluconic acid, glucuronic acid, saccharic acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid. Therefore, the salt can be a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisate salt, a gluconate salt, a glucuronate salt, a saccharate salt, a formate salt, a benzoate salt, a glutamate salt, a pantothenate salt, an acetate salt, a propionate salt, a butyrate salt, a fumarate salt, a succinate salt, a methanesulfonate salt, an ethanesulfonate salt, a benzenesulfonate salt, a p-toluenesulfonate salt, a citrate salt, an oxalate salt, or a maleate salt.

Prodrugs can include an amino acid covalently attached to a psychoactive base substance of MDMA or an MDMA-like compound. The addition of the amino acid makes the active compound inactive mainly by preventing interaction with monoamine transporter, which is the site of action but also affecting bioavailability/rate of absorption. The amino acid can be lysine or any other amino acid such as alanine, arginine, asparagine, aspartic acid, cysteine, glutamine, glutamic acid, glycine, histidine, isoleucine, leucine, methionine, phenylalanine, proline, serine, threonine, tryptophan, tyrosine, or valine and typically attached to the amine (N)-group of MDMA or the MDMA-like substance and hence reducing pharmacological activity at the primary site of action (cell-membrane monoamine transporters including serotonin, dopamine and norepinephrine transporter), and also altering extent and rate of absorption and mainly releasing active substance in the circulation after absorption of the inactive compound. The amino acid can be any other natural or synthetic amino acid. The amino acid can be covalently bound to a MDMA-like substance via the amine group of the MDMA-like substance to form a peptide bond.

Doses of any of the compounds herein can be 20-300 mg. Specifically, in humans, R-MDMA can be administered at 50 mg to 300 mg, and in particular at the higher dose of 300 mg, is expected to result in greater psychedelic-type effects (5D-ASC total OAV score) compared with S-MDMA (125 mg). R-MDMA, administered at 75 mg, 125 mg, 175 mg or 225 mg resulted in a differentiated subjective experience compared to racemic MDMA on standard psychedelic experience questionnaires. Further, dose dependent changes on measures of emotional breakthrough, a phenomenon thought to be a key mediator of the long-term psychological changes associated with psychedelics, were noted in this healthy volunteer population. Compounds can be administered a single dose or multiple doses, up to three times per day. In contrast, 125 mg of S-MDMA is postulated to induce greater subjective stimulation (VAS) than 125 mg of R-MDMA. The compounds can be administered before, during, or after the individual has the sensation of pain.

While the mechanism of the compounds with respect to pain is not clear, they can act peripherally and centrally and provide a psychological effect as well as a direct neural effect to treat pain. The compounds of the present invention can more rapidly treat pain, provide longer-lasting relief, and provide more pain reduction compared to NSAIDs, opioids and SSRIs. The compounds of the present invention can be administered in such a way to treat pain but without unwanted side effects. The compounds can also alter the individual's mood to reduce and relieve anxiety that can cause and simultaneously result in pain, in addition to the direct pain reducing effect. EXAMPLES 1 and 2 show that R-MDMA can provide more pain tolerance in a mouse tail flick test and carrageenan-induced hyperalgesia.

The methods of the present invention can further include administering an anti-inflammatory and/or anti-pain agent before, during, or after administration of the MDMA compounds described above. The compound and the anti-inflammatory/anti-pain agent can be in the same single dosage form or different dosage forms. The anti-inflammatory/anti-pain agent can be non-steroidal anti-inflammatory drugs (NSAIDS), such as, but not limited to, acetaminophen, salicylates (aspirin, diflunisal, salsalate), acetic acid derivatives (indomethacin, ketorolac, sulindac etodolac, diclofenac, nabumetone), propionic acid derivatives (ibuprofen, naproxen, flurbiprofen, ketoprofen, oxaprozin, fenoprofen, loxoprofen), fenamic acid derivatives (meclofenamic acid, mefenamic acid, flufenamic acid, tolfenamic acid), oxicam (enolic acid) derivatives (piroxicam, meloxicam, tenoxicam, droxicam, lornoxicam, isoxicam), arylalkanoic acid derivatives (tolmetin); or selective COX-2 inhibitors (celecoxib, rofecoxib, valdecoxib, parecoxib, lumiracoxib, etoricoxib, firocoxib). The anti-inflammatory/anti-pain agent can also be steroids such as, but not limited to, corticosteroids (hydrocortisone, hydrocortisone acetate, cortisone acetate, tixocortol pivalate, prednisolone, methylprednisolone, prednisone, triamcinolone acetonide, triamcinolone alcohol, mometasone, amcinonide, budesonide, desonide, fluocinonide, fluocinolone acetonide, halcinonide, betamethasone, dexamethasone, fluocortolone, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, or fluprednidene acetate). The anti-inflammatory/anti-pain agent can further be immune selective anti-inflammatory derivatives (ImSAIDs) such as, but not limited to, submandibular gland peptide T (SGp-T) and derivatives phenylalanine-glutamine-glycine (FEG) and its D-isomeric form (feG).

The anti-inflammatory/anti-pain agent can also be a narcotic composition such as, but not limited to, buprenorphine, butorphanol, codeine, hydrocodone, hydromorphone, levorphail, meperidine, methadone, morphine, nalbuphine, oxycodone, oxymorphone, pentaxocine, or propoxyphene.

The anti-inflammatory/anti-pain agent can also be other analgesic compositions such as, but not limited to, tramadol, or capsaicin. The anti-inflammatory/anti-pain agent can also be a topical anesthetic, such as, but not limited to, benzocaine, dibucaine, lidocaine, or prilocaine.

The anti-inflammatory/anti-pain agent can also be any suitable biologic agent that reduces inflammation and/or pain, such as etanercept (ENBREL®, Amgen, Inc.). Etanercept reduces the levels of inflammatory-causing tumor necrosis factor (TNF) in the body and is administered by injection. Other biologic agents that inhibit IL-1 or TNF, or that effect other biologic pathways, can also be used, such as, but not limited to, adalimumab (HUMIRA®, Abbott), anakinra (KINERET®, Amgen, Inc.), infliximab (REMICADE®, Janssen Biotech, Inc.), certolizumab-pegol (CIMZIA®, UCB, Inc.), and Natalizumab (TYSABRI®, Biogen Idec).

The anti-inflammatory/anti-pain agent can further be any combination of the above compositions along with other agents. Some readily available combinations of anti-inflammatory/anti-pain agents are as follows: butalbital, acetaminophen, and caffeine; butalbital, aspirin, and caffeine; butalbital, acetaminophen, caffeine, and codeine; hydrocodone and ibuprofen; pentazocine and naloxone; acetaminophen and codeine; dihydrocodeine, acetaminophen, and caffeine; hydrocodone and acetaminophen; oxycodone and acetaminophen; pentazocine and acetaminophen; propoxyphene and acetaminophen; aspirin, caffeine, and dihydrocodeine; aspirin and codeine; hydrocodone and aspirin; oxycodone and aspirin; pentazocine and aspirin; and propoxyphene, aspirin, and caffeine.

In addition to the administration methods listed below, the compounds can also be provided in dosage forms that are more amenable to treating pain, such as, but not limited to, transdermal patches, modified-release oral dosage forms, extended release injection, implanted titration device, intranasal delivery forms, or sublingual delivery forms.

The compounds of the present invention are administered and dosed in accordance with good medical practice, considering the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compounds of the present invention can be administered in various ways. It should be noted that they can be administered as the compound and can be administered alone or as an active ingredient in combination with pharmaceutically acceptable carriers, diluents, adjuvants, and vehicles. The compounds can be administered orally, transcutaneously, subcutaneously or parenterally including intravenous, intramuscular, and intranasal administration. The patient being treated is a warm-blooded animal and, in particular, mammals including man. The pharmaceutically acceptable carriers, diluents, adjuvants, and vehicles as well as implant carriers generally refer to inert, non-toxic solid or liquid fillers, diluents or encapsulating material not reacting with the active ingredients of the invention.

The doses can be single doses or multiple doses or a continuous dose over a period of several hours, days, weeks, months, or years.

When administering the compound of the present invention parenterally, it will generally be formulated in a sublingual or buccal dissolving tablet, dissolving film, intranasal powder, intranasal solution, inhaled powder, inhaled solution, transdermal patch, transdermal patch with microneedles or other permeation enhancers, or as a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

The invention is further described in detail by reference to the following experimental examples. These examples are provided for the purpose of illustration only, and are not intended to be limiting unless otherwise specified. Thus, the invention should in no way be construed as being limited to the following examples, but rather should be construed to encompass any and all variations which become evident as a result of the teaching provided herein.

### Example 1

### Acute Pain: Mouse Tail Flick

### Method

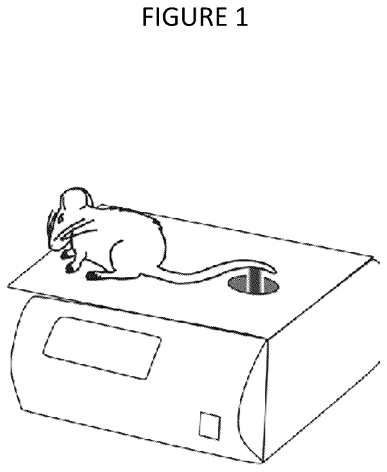

Mouse Tail Flick is a model to test antinociception in animals. Mice were acclimated to the procedure room for at least 30 minutes prior to the experiment. Ambient room temperature was closely monitored (22-23° C.). Mice were tested on the radiant tail flick apparatus (Columbus Instruments). The heat was adjusted so as to give 3-5 second baseline latencies. Each mouse was restrained and the distal half of the tail placed above the heat source. Tail flick latency was measured as the time from the onset of the heat exposure to the time of withdrawal of the tail. Tail withdrawal latency was recorded with a cut-off time of 10 seconds. A set of trials was made and averaged at each time point. A maximum score is assigned to animals not responding within the cut-off time, to avoid tissue damage. The apparatus is illustrated in FIG. 1.

### Experimental Design

Route(s) of administration: PO
Dose Volume(s): 10 ml/kg
Formulation(s): PBS
Dose Levels: R-MDMA 17 and 30 mg/kg, Salt CF=1.19
Dose Frequency: 1×
Study duration: 1 day
Number of Groups: 4
Number of animals per group: 10
Total number of animals: 40
TABLE 1 shows the experimental design.

TABLE 1

| group | | Group | Days of | | | Evaluations/ |
|---|---|---|---|---|---|---|
| # | TReatment | Size | Dosing | DOSe | Route | Endpoints |
| 1 | Vehicle | 10 | 1 | 0 | PO | Tail Flick |
| 2 | R-MDMA - | | | 17 mg/kg | | (latency to |
| | 17 mg/kg | | | | | nociceptive |
| 3 | R-MDMA- | | | 30 mg/kg | | response) |
| | 30 mg/kg | | | | | 30 minutes |
| 4 | Morphine | | | 4 mg/kg | SC | 60 minutes |
| | | | | | | 90 minutes |

### Results

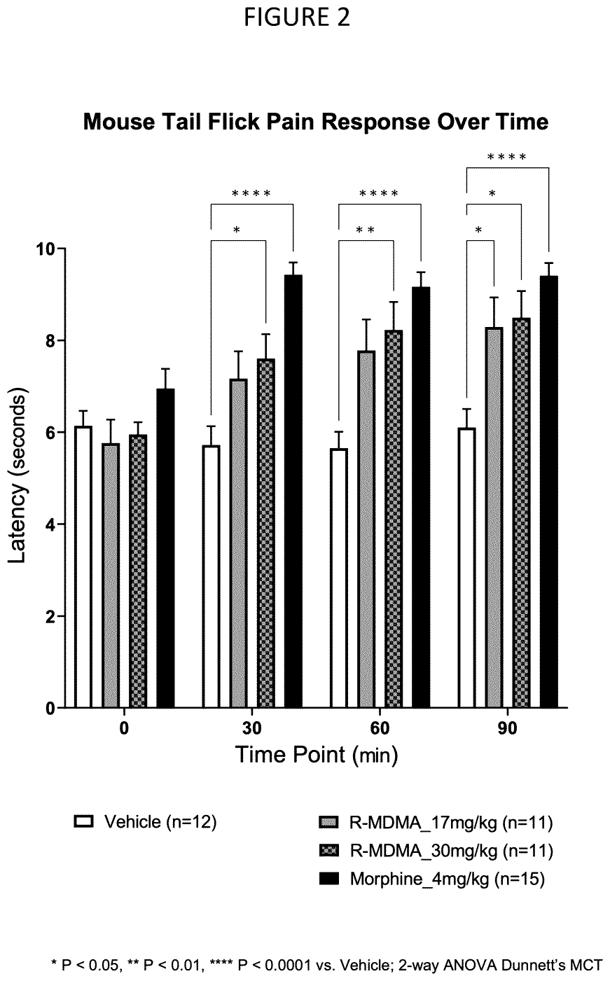

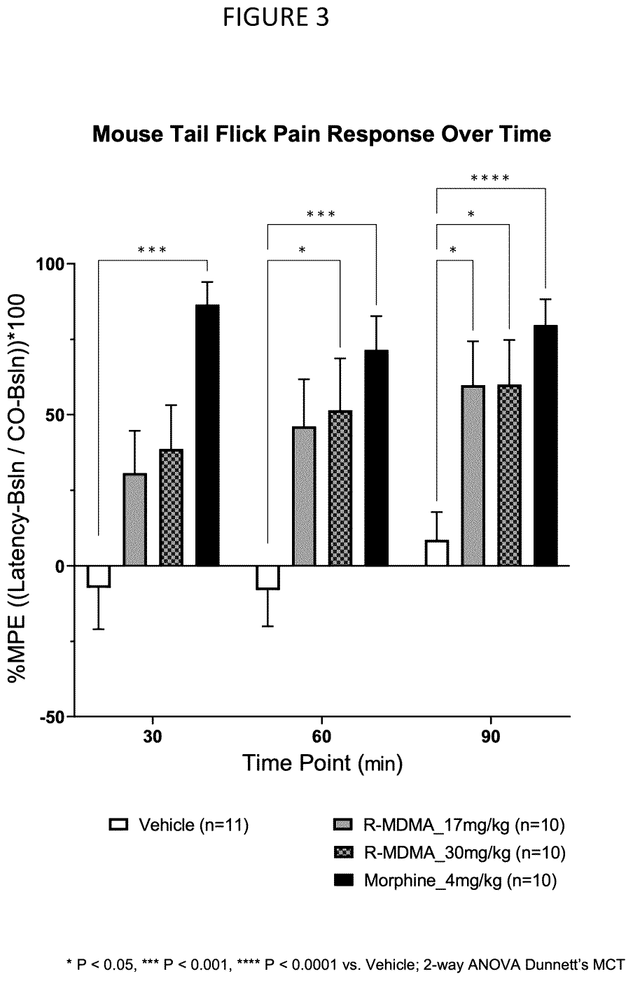

R-MDMA administered at 17 and 30 mg/kg PO produced a statistically significant increase in the latency of the tail flick comparable to that of morphine at 30, 60 and 90 minutes after the administration (FIG. 2). Percent of maximal possible effect (MPE) is calculated. The % MPE was calculated as [(T1-T0)/(T2-T0)]×100. T0 and T1 were the tail flick latencies before and after drug administration, and T2 was the cut off time. % of maximal possible effect (MPE) is calculated. % MPE was significantly increased in R-MDMA treated animals at 60 and 90 minutes post-treatment. R-MDMA is active in the model of acute pain with MPE 60% (compared to 80% for morphine) (FIG. 3).

### Example 2

### Inflammatory Pain: Carrageenan-Induced Hyperalgesia

### Method

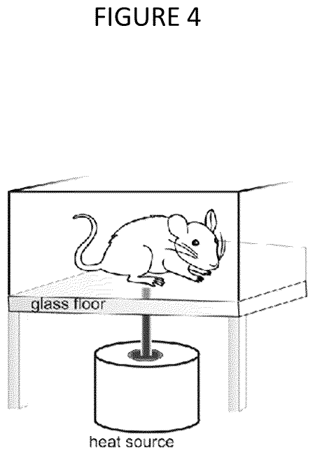

After baseline Hargreaves testing, rats receive an intraplantar injection of 2% carrageenan into the left hindpaw and replaced in home cage until acclimation/testing. See FIG. 4 below for example event order following baseline testing.

Rats were dosed PO with vehicle, reference compound (ibuprofen), or test compound 180 minutes after administration of carrageenan (T180 min).

At 60 minutes and 120 minutes post TA dose (T240 min, T300 min), thermal hyperalgesia was measured using the Hargreaves test.

Rats were examined for thermal pain thresholds following intraplantar carrageenan injection. Rats were acclimated to the procedure room for at least 30 minutes prior to the experiment. Ambient room temperature was closely monitored around 22-23° C. To assess the thermal sensitivity of the hind paw, rats were placed on the glass surface of a thermal radiant heat testing apparatus (Hargreaves apparatus). The rats were allowed to acclimate to the apparatus for 30 min before testing. The temperature of the glass surface is maintained constant at 28° C. A mobile radiant heat source located under the glass was focused onto the hind paw of each rat. The apparatus was adjusted at the beginning of the study so that the baseline paw withdrawal latency in normal rats were approximately 10 seconds. This setting (the light beam intensity) then was used throughout the study. A cutoff of 20 seconds was used to prevent potential tissue damage. FIG. 4 shows an example of the testing apparatus.

### Experimental Design

Route(s) of administration:
Carrageenan: Intraplantar subcutaneous-2%
TA: PO
Dose Volume(s): 3 mL/kg
Formulation(s): PBS
Dose Levels
TA2, R-MDMA 12.5 and 25 mg/kg; Salt CF=1.19
Dose Frequency: QD 1 day
Number of Groups: 4
Number of animals per group: 10
Total number of animals: 40

The experimental design is shown in TABLE 2.

TABLE 2

| group | | Group | Days of | | | Evaluations/ |
|---|---|---|---|---|---|---|
| # | TReatment | Size | Dosing | DOSe | Route | Endpoints |
| 1 | Vehicle; | 10 | 1 | 0 | PO | Hargreaves |
| | QD - 7 days | | | | | Test (time |
| 2 | R-MDMA; | | 1 | 12.5 | | relative to |
| | 12.5 mg/kg | | | | | R-MDMA |
| | acute | | | | | administra- |
| 3 | R-MDMA; | | | 25 | | tion) (Hy- |
| | 25 mg/kg | | | | | peralgesia |
| | acute | | | | | sensitivity |
| 4 | Ibuprofen | | | 100 mg/kg | | to thermal |
| | | | | | | stimulus) |
| | | | | | | Baseline |
| | | | | | | 60 minutes |
| | | | | | | 120 minutes |

### Results

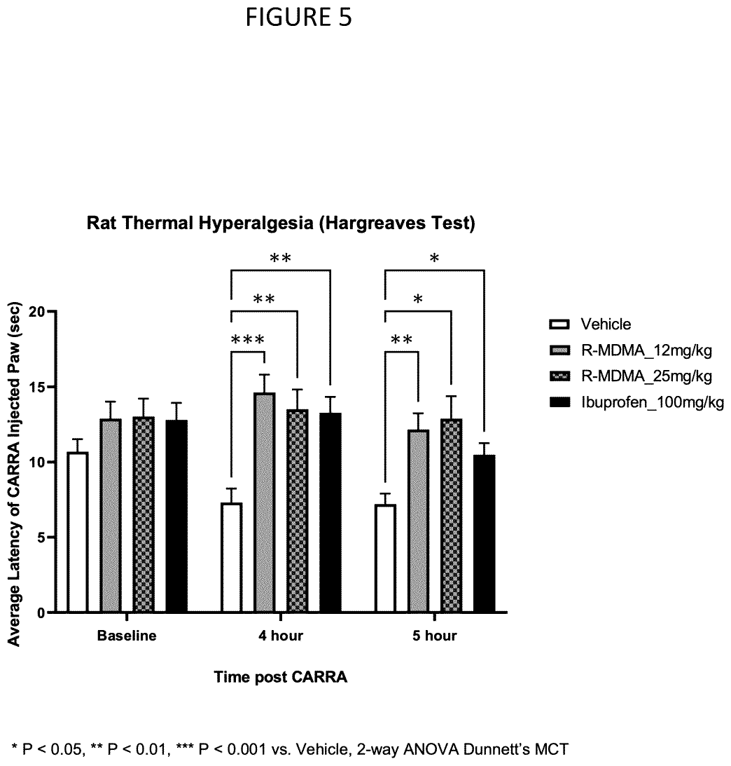

Administration of R-MDMA 3 hours post-carrageenan resulted in a significant decrease of thermal hyperalgesia. The latency to withdraw the injured paw was significantly increased compared to the vehicle treatment (P<0.001, P<0.01), as demonstrated in FIG. 5.

Throughout this application, various publications, including United States patents, are referenced by author and year and patents by number. Full citations for the publications are listed herein. The disclosures of these publications and patents in their entireties are hereby incorporated by reference into this application in order to more fully describe the state of the art to which this invention pertains.

The invention has been described in an illustrative manner and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A method of treating pain, including the steps of:
administering an effective amount of a composition chosen from the group consisting of MDMA, R-MDMA, S-MDMA, a mixture of enantiomers not in a 1:1 ratio, an MDMA-like compound, a prodrug of MDMA, a prodrug of R-MDMA, a prodrug of S-MDMA, a prodrug of an MDMA-like compound, analogs thereof, derivatives thereof, and salts thereof to an individual; and
treating pain in the individual.

2. The method of claim 1, wherein the pain is chosen from the group consisting of acute, chronic, nociceptive, neuropathic, inflammatory, and functional.

3. The method of claim 1, wherein the pain is caused by a physical state in the individual's body.

4. The method of claim 1, wherein the pain is caused by an emotional state in the individual's body.

5. The method of claim 1, wherein the MDMA-like compound is chosen from the group consisting of 3,4-methylenedioxyamphetamine (MDA), 3,4-methylenedioxyethylamphetamine (MDEA), 1-(1,3-benzodioxol-5-yl)methyl-2-butanamine (MBDB), 1-(1,3-benzodioxol-5-yl)-2-aminobutane (BDB, also known as MDB) methylone, ethylone, 5,6-methylenedioxy-2-aminoindane (MDAI), 5-iodo-2-aminoindane (5-IAI), 4-(2-aminopropyl)-benzofuran (4-APB), 5-(2-aminopropyl)-benzofuran (5-APB), 6-(2-aminopropyl)-benzofuran (6-APB), N-methyl-1-(2,3-dihydrobenzofuran-5-yl)-propan-2-amine (5-MAPDB), and 6-(2-methylaminopropyl)-benzofuran (6-MAPB).

6. The method of claim 1, wherein the salt is chosen from the group consisting of a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, a zinc salt, a triethyl amine salt, trimethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrazole salt, a pyridazine salt, a pyrimidine salt, an imidazole salt, a pyrazine salt, a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisate salt, a gluconate salt, a glucuronate salt, a saccharate salt, a formate salt, a benzoate salt, a glutamate salt, a pantothenate salt, an acetate salt, a propionate salt, a butyrate salt, a fumarate salt, a succinate salt, a methanesulfonate salt, an ethanesulfonate salt, a benzenesulfonate salt, a p-toluenesulfonate salt, a citrate salt, an oxalate salt, and a maleate salt.

7. The method of claim 1, wherein the prodrug includes an amino acid covalently attached to a psychoactive base substance of the composition.

8. The method of claim 7, wherein the amino acid is chosen from the group consisting of lysine, alanine, arginine, asparagine, aspartic acid, cysteine, glutamine, glutamic acid, glycine, histidine, isoleucine, leucine, methionine, phenylalanine, proline, serine, threonine, tryptophan, tyrosine, and valine.

9. The method of claim 1, wherein said administering step is further defined as administering 20-300 mg of the composition.

10. The method of claim 1, wherein said administering step is performed at a time chosen from the group consisting of before, during, and after the individual has a sensation of pain.

11. The method of claim 1, wherein said treating step is further defined as the composition providing a psychological effect and a direct neural effect in treating pain.

12. The method of claim 1, further including the step of administering an anti-inflammatory/anti-pain agent at a time chosen from the group consisting of before, during, and after said administering step.

13. The method of claim 12, wherein the anti-inflammatory/anti-pain agent is chosen from the group consisting of non-steroidal anti-inflammatory drugs (NSAIDS), selective COX-2 inhibitors, steroids, immune selective anti-inflammatory derivatives (ImSAIDs), a narcotic composition, analgesic compositions, a topical anesthetic, a biologic agent, and combinations thereof.

14. The method of claim 12, wherein said anti-inflammatory/anti-pain agent is in the same single dosage form with the composition.
